(19) United States
(12) Patent Application Publication
(10) Pub. No.: US 2005/0176752 A1
(43) Pub. Date: Aug. 11, 2005

(54) Process for the preparation of piperidine derivatives

(76) Inventors: Jean-Francois Meunier, Montreal (CA); Herika Marrugo, Verdun (CA); Gregory Bydlinski, Montreal (CA)

Correspondence Address: Richard J. Streit;Ladas & Parry LLP, Suite 1200, 224 South Michigan Avenue, Chicago, IL, 60604, US

(21) Appl. No.: 11/031,056

(22) Filed: Jan. 7, 2005

(30) Foreign Application Priority Data
Jan. 9, 2004 (CA) 2,455,018

(51) Int. Cl. A61K 31/4741; C07D 491/02

(52) U.S. Cl. 514/302; 546/114

(57) ABSTRACT

A process is disclosed for preparation of piperidine derivatives, preferably 1,3-dioxolo [4,5-c] piperidin-7-ols such as (3aS, 4R, 7S, 7aR)-2,2,4-trimethyl-1,3-dioxolo [4,5-c] piperidin-7-ol and its polyhydroxylated derivatives. In a preferred process, 2,3-O-isopropylidene-1,4-lactone (A) is reacted with methanesulfonyl chloride to form (3aR, 4S, 6aR) methanesulfonic acid 2,2-dimethyl-6-oxo-tetrahydro-furo[3,4-d][1,3]dioxol-4-ylmethyl (B). Compound (B) is then reacted with methylmagnesium halide to form (3aR, 4S, 6aR)-methanesulfonic acid 6-hydroxy-2,2,6-trimethyl-tetrahydro-furo[3,4-d][1,3]dioxol-4-ylmethyl ester (C), which is reacted with phthalimide to form (3aR, 4S, 6aR)-2-(6-hydroxy-2,2,6-trimethyl-tetrahydro-furo[3,4-d][1,3]dioxol-4-ylmethyl)-isoindole-1,3-dione (D). Compound (D) is reacted with hydrazine to form (3S, 7S, 7aR)-2,2,4-trimethyl-3a,6,7,7a-tetrahydro-[1,3]dioxolo[4,5-c]pyridin-7-ol (E), which is hydrogenated to give the corresponding 1,3-dioxolo [4,5-c] piperidin-7-ol (F). The synthesis has an overall yield which is typically greater than 50% and avoids the use of reagents such as triflic anhydride and sodium azide.

### FIELD OF THE INVENTION

The invention relates to processes for the preparation of piperidine derivatives and more particularly to processes for preparing as 1,3-dioxolo [4,5-c] piperidin-7-ols and related compounds.

### BACKGROUND OF THE INVENTION

1,3-Dioxolo [4,5-c] piperidin-7-ols such as the compound (3aS, 4R, 7S, 7aR)-2,2,4-trimethyl-1,3-dioxolo [4,5-c] piperidin-7-ol and its polyhydroxylated derivatives are an important group of molecules having various biological properties.

Recently, researchers have discovered a new group of therapeutic iminosugars which were found to be effective for treating pestivirus and flavivirus infections such as hepatitis B, hepatitis C, dengue and Japanese encephalitis. Examples of these iminosugars and processes for their preparation are described in International Patent Application No. PCT/US00/21732 filed Aug. 10, 2000; published on Feb. 15, 2001 as WO 01/10429, which is incorporated herein by reference in its entirety.

A number of the virus-inhibiting compounds disclosed by International Publication No. WO 01/10429 are piperidine derivatives which can be obtained from corresponding N-alkyl or N-oxa-alkyl 1,3-dioxolo [4,5-c] piperidin-7-ols. Examples of such 1,3-dioxolo [4,5-c] piperidin-7-ols are N-nonyl-1,5,6-trideoxy-1,5-imino-3,4-O-isopropylidene-D-galactitol; N-(7-oxa-nonyl)-1,5,6-trideoxy-1,5-imino-3,4-O-isopropylidene-D-galactitol; N-nonyl-1,5-dideoxy -1,5-imino-3,4-O-isopropylidene-D-galactitol; and N-(7-oxa-nonyl)-1,5-dideoxy -1,5-imino-3,4-O-isopropylidene-D-galactitol. These compounds can be used to prepare the virus-inhibiting compounds referred to in WO 01/10429 as N-nonyl MeDGJ, N-7-oxa-nonyl MeDGJ, N-nonyl DGJ and N-7-oxa-nonyl DGJ, respectively.

A process for the preparation of (3aS, 4R, 7S, 7aR)-2,2,4-trimethyl-1,3-dioxolo [4,5-c] piperidin-7-ol is also disclosed by International Publication No. WO 01/10429. This compound is prepared by a six-step synthesis beginning from D-gulonolactone. The six steps in the synthesis are described at page 17, line 4 to page 21, line 19 of this publication. In step 4 of the synthesis, the hydroxyl group of 2,3-O-isopropylidene-L-lyxono-1,4-lactone is replaced by an azide group by reacting 2,3-O-isopropylidene-L-lyxono-1,4-lactone first with trifluoromethanesulfonic anhydride (referred to herein as “triflic anhydride”) and then with sodium azide. Due to the highly reactive and corrosive nature of triflic anhydride and the high toxicity of sodium azide, the handling of these two reagents and the corresponding synthetic intermediates is technically demanding. Furthermore, the cost of triflic anhydride reagent and the synthesis as a whole are relatively high.

There is a need for a process for preparing 1,3-dioxolo [4,5-c] piperidin-7-ols and related compounds which avoids use of reagents such as triflic anhydride and sodium azide.

### SUMMARY OF THE INVENTION

The present invention provides a process for preparing piperidine derivatives which avoids use of reagents such as triflic anhydride and sodium azide, thereby avoiding some of the special handling involved in working with these reagents and making the process of the invention more attractive for use on an industrial scale. The process of the invention is particularly useful for preparing 1,3-dioxolo [4,5-c] piperidin-7-ols such as (3aS, 4R, 7S, 7aR)-2,2,4-trimethyl -1,3-dioxolo [4,5-c] piperidin-7-ol and its polyhydroxylated derivatives.

In a preferred aspect, the present invention provides a five-step synthesis for preparing (3aS, 4R, 7S, 7aR)-2,2,4-trimethyl-1,3-dioxolo [4,5-c] piperidin-7-ol from 2,3-O-isopropylidene-L-lyxono-1,4-lactone. A particularly preferred embodiment of this process, showing preferred reagents, is depicted below in Scheme I.

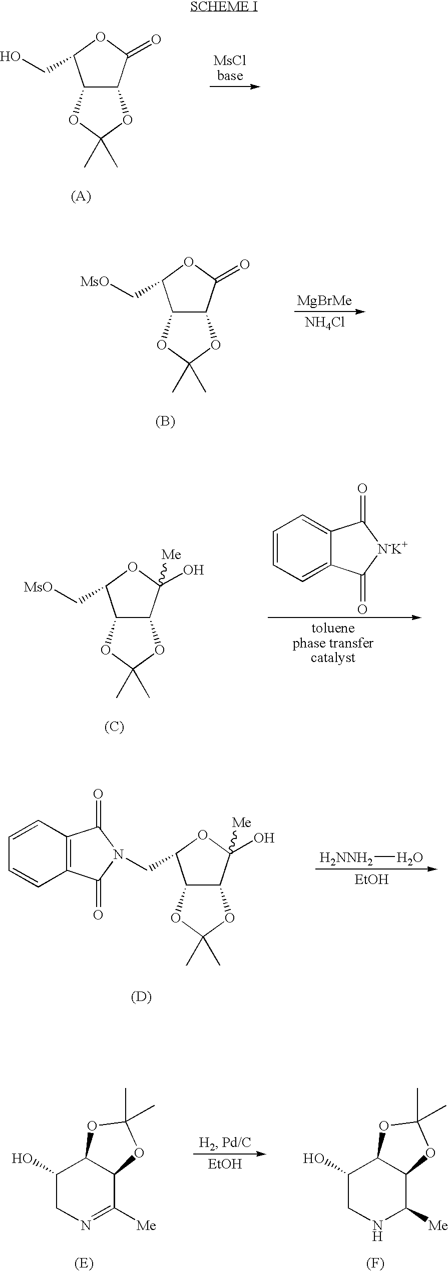

The process according to the present invention is also useful to prepare other 1,3-dioxolo [4,5-c] piperidin-7-ols and may employ reagents other than those depicted above in Scheme I. A more general process within the scope of the present invention is illustrated below in Scheme II.

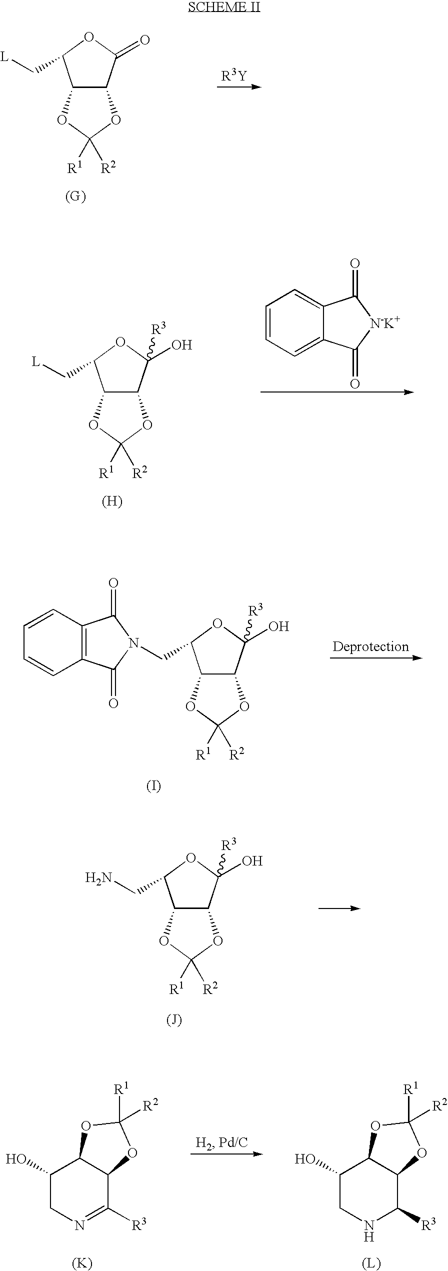

In the process shown in Scheme II, L is any suitable leaving group, R³Y is an alkylating agent, and R¹, R² and R³ are independently selected from the group comprising hydrogen, alkyl and alkoxy. Where these groups are alkyl or alkoxy groups, they are preferably lower alkyl or lower alkoxy containing from 1 to 4 carbon atoms.

### DETAILED DESCRIPTION OF PREFERRED EMBODIMENTS

The following are detailed descriptions of preferred processes according to the invention for preparing 1,3-dioxolo [4,5-c] piperidin-7-ols such as (3aS, 4R, 7S, 7aR)-2,2,4-trimethyl-1,3-dioxolo [4,5-c] piperidin-7-ol and its polyhydroxylated derivatives.

The starting material (G) in the general process illustrated in Scheme II is 2,3-O-isopropylidene-L-lyxono-1,4-lactone which is modified by insertion of a leaving group L. Group L can be any suitable leaving group, including halides, sulfonates and acetates. Preferred halides include chloride, bromide and iodide ions and preferred sulfonates include mesylate, tosylate, brosylate and triflate ions. Triflate is, however, less preferred since it is introduced into compound (G) by triflic anhydride, which is preferably avoided. The triflate ion can also be introduced by trifluoromethanesulfonyl chloride.

In a particularly preferred embodiment of the invention, 2,3-O-isopropylidene -L-lyxono-1,4-lactone (A) is reacted with mesyl chloride (MsCl) in the presence of a base to provide (3aR, 4S, 6aR)-methanesulfonic acid 2,2-dimethyl -6-oxo-tetrahydro-furo[3,4-d][1,3]dioxol-4-ylmethyl (B). This step is shown below.

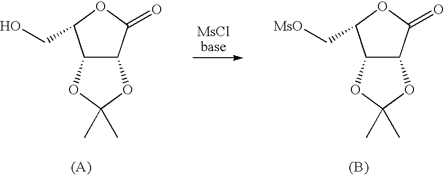

The reaction of compound (A) with mesyl chloride is conducted in an inert solvent which may preferably comprise one or more of ethyl acetate, isopropyl acetate, acetone, methyl ethyl ketone, methyl isobutyl ketone, dichloromethane, ethyl ether, t-butylmethyl ether, tetrahydrofuran, toluene and any other suitable inert solvent. Any suitable base can be used, including amines such as triethylamine, pyridine and carbonates such as potassium carbonate. Compound (B) is obtained as a white to off-white solid precipitate in a yield of from about 85% to about 95%.

As shown below in Scheme III, 2,3-O-isopropylidene-L-Iyxono-1,4-lactone (A) can be formed from commercially available D-gulonolactone (M) in the following three steps:

(1) Formation of acetonide group to give 2,3,5,6-Di-O-isopropylidene-D-gulono-1,4-lactone (N);

(2) Deprotection of the 1,2-diol function to give 2,3-O-isopropylidene-D-gulono-lactone (O); and

(3) Oxidative cleavage with periodic acid to give compound (A).

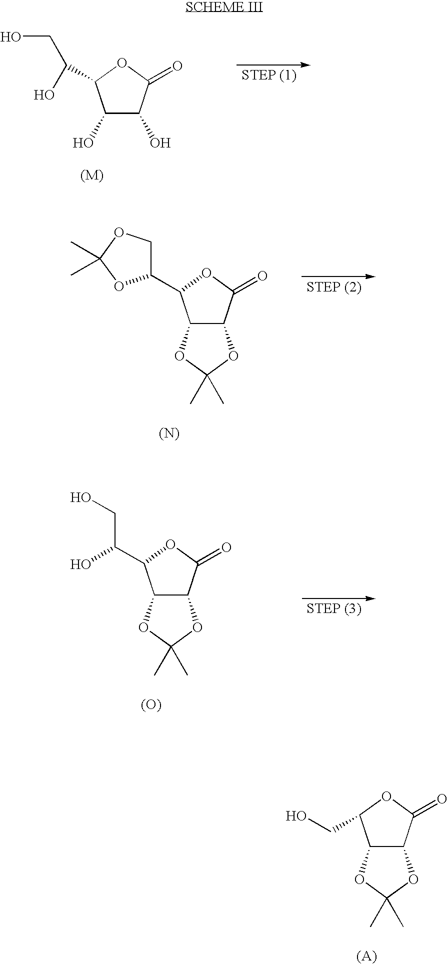

A similar three-step synthesis for preparing compound (A) from D-gulonolactone is described at pages 17 and 18 of International Publication No. WO 01/10429.

Returning to the general synthesis of Scheme II, after insertion of the leaving group to form compound (G), the next step in the synthesis involves alkylation of the 6-carbonyl group of compound (G) to form the hemiacetal group of compound (H), which is a protected, 6-alkylated form of 2,3-O-isopropylidene-L-lyxono -1,4-lactone (A).

The alkylating agent shown in Scheme I is R³Y. R³ is preferably an alkyl or alkoxy group, more preferably a lower alkyl or lower alkoxy group having from 1 to 4 carbon atoms. More preferably, R³ is a methyl or methoxy group, and most preferably R³ is a methyl group. Y is preferably a magnesium halide group such as —MgBr, —MgCl or —MgI or a metal ion such as lithium ion. Particularly preferred alkylating agents are CH₃MgBr, CH₃MgCl, CH₃MgI and CH₃Li.

The alklylation reaction is performed in an inert solvent selected from one or more of toluene, THF, ethyl ether, tert-butylmethyl ether, etc. The reaction temperature ranges from −10° C. to 25° C. and a small excess of reagent is preferably used, preferably about 1.1 molar equivalent. The work-up of the reaction needs a proton source like ammonium chloride or any other dilute acid.

In the preferred alkylation step of Scheme I shown below, (3aR, 4S, 6aR)-methanesulfonic acid 2,2-dimethyl-6-oxo-tetrahydro-furo[3,4-d][1,3]dioxol-4-ylmethyl (B) is reacted with methylmagnesium bromide and is worked up with ammonium chloride to yield (3aR, 4S, 6aR)-methanesulfonic acid 6-hydroxy-2,2,6-trimethyl-tetrahydro-furo[3,4-d][1,3]dioxol-4-ylmethyl ester (C) as a white to off-white solid in a typical yield of from about 85% to about 95%.

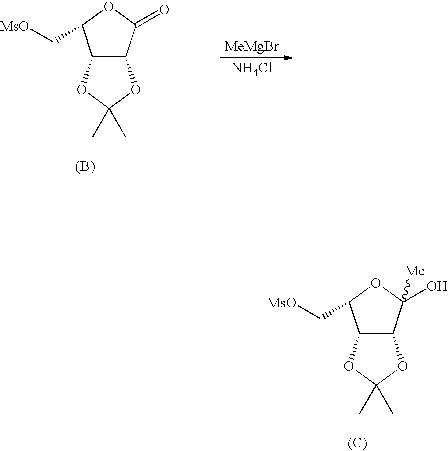

The next step in the general synthesis of Scheme II comprises an amination step in which the leaving group L is replaced by a phthalimide group. The phthalimide group is introduced by reacting compound (H) with potassium phthalimide or sodium phthalimide. The reaction is preferably conducted in an organic solvent such as toluene with a suitable phase transfer catalyst, or in an aprotic solvent such as dimethylformamide (DMF) or dimethylsulfoxide (DMSO). It will be appreciated that the order of the alkylation and the amination steps can be reversed, such that amination of compound (G) with potassium phthalimide is followed by methylation of the lactone to give compound (I).

In the preferred process shown in Scheme I, methylation of lactone (B) with methylmagnesium bromide in THF is followed by displacement of the mesylate group with potassium phthalimide in hot toluene and with hexadecyltributylphosphonium bromide as the phase transfer catalyst. As shown below, the product of the amination in the preferred process of Scheme I is (3aR, 4S, 6aR)-2-(6-hydroxy-2,2,6-trimethyl-tetrahydro-furo[3,4-d][1,3]dioxol-4-ylmethyl) -isoindole-1,3-dione (D) which is usually obtained in a yield of from about 75 to about 80%. The formation of product (D) from compound (C) proceeds through intermediate (D′), 4R-aceto-2,2-dimethyl-5R-(2S-oxiranyl)-[1,3]-dioxolane.

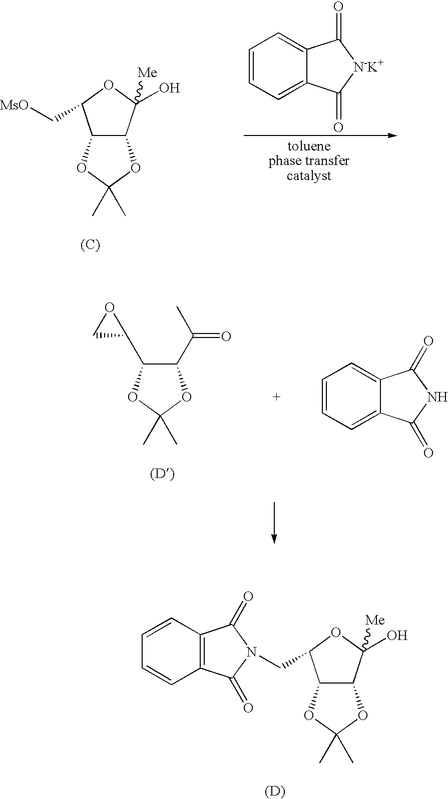

The next step in the general process of Scheme II is deprotection of the amine function by cleavage of the phthalimide group of compound (I). Cleavage of the phthalimide group is accomplished by any one of a number of methods, such as by hydrazine, methylamine, n-pentylamine, sodium sulphide or by alkaline hydrolysis. The cleavage is preferably performed by reacting compound (I) with hydrazine monohydrate in refluxing ethanol or another solvent such as isopropanol. As shown in Scheme II, the deprotected amino compound (J) immediately undergoes cyclization to form the tetrahydropyridinol compound (K). The crude reaction product is preferably used directly in the next step without isolation or purification.

As shown below, in the preferred process of Scheme I the (3aR, 4S, 6aR)-2-(6-hydroxy-2,2,6-trimethyl-tetrahydro-furo[3,4-d][1,3]dioxol-4-ylmethyl) -isoindole-1,3-dione (D) is reacted with hydrazine monohydrate in refluxing ethanol to give (3S, 7S, 7aR)-2,2,4-trimethyl-3a,6,7,7a-tetrahydro-[1,3]dioxolo[4,5-c]pyridin-7-ol (E).

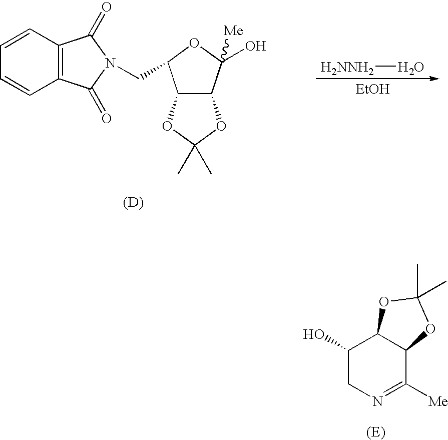

The tetrahydropyridinol products (E) and (K) obtained by Schemes I and II, respectively are preferably hydrogenated to convert the tetrahydropyridine ring to a piperidine ring. In the general process of Scheme II, the final product is compound (L). In the preferred process, hydrogenation of compound (E) yields (3aS, 4R, 7S, 7aR)-2,2,4-trimethyl-1,3-dioxolo [4,5-c] piperidin-7-ol (F), as shown below.

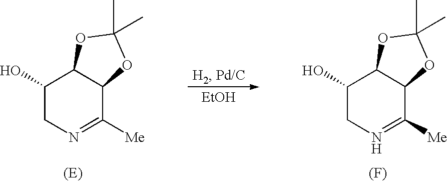

The hydrogenation is preferably performed in a hydrogenation reactor at 25° C. to 50° C. with a pressure between 45-50 psi of hydrogen and a catalytic amount of palladium on charcoal. The solvent is preferably ethanol from the deprotection step, however it will be appreciated that other solvents can be used for this reaction. A white to off-white solid precipitate is easily isolated with a combined yield of from about 80% to about 90% for the deprotection and hydrogenation steps.

Thus, the present invention enables the preparation of highly pure (3aS, 4R, 7S, 7aR)-2,2,4-trimethyl-1,3-dioxolo [4,5-c] piperidin-7-ol (F) via easily isolable, stable, solid intermediates and an overall yield which is typically greater than 50%.

It will be appreciated that the final product of the process, i.e. compound (L) of the general process or compound (F) of the preferred process, may be further reacted to produce virus-inhibiting compounds (P), which are of the same general structure as compounds described in International Publication No. WO 01/10429. The compounds of formula (P) are also referred to herein as the “polyhydrolylated derivatives” of 1,3-Dioxolo [4,5-c] piperidin-7-ols such as (3aS, 4R, 7S, 7aR)-2,2,4-trimethyl-1,3-dioxolo [4,5-c] piperidin-7-ol.

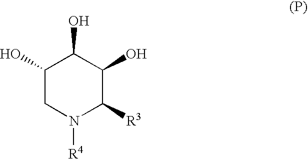

In formula (P), the R⁴ group is a C₈ to C₁₆ alkyl and is optionally substituted with 1 to 5, preferably 1 to 3, and more preferably 1 to 2 oxygen atoms (i.e. oxa-substituted). Preferably, the R⁴ group is a C₈ to C₁₀ alkyl or oxa-alkyl group. Particularly preferred alkyl groups are n-nonyl and n-decyl. Preferred oxa-alkyl groups are 3-oxanonyl, 3-oxadecyl, 7-oxanonyl and 7-oxadecyl.

The invention is further illustrated by the following non-limiting examples. All procedures were carried out under an inert atmosphere (nitrogen was used in the examples).

### EXAMPLES

### Example 1

### Formation of (3aR, 4S, 6aR) methanesulfonic acid 2,2-dimethyl -6-oxo-tetrahydro-furo[3,4-d][1,3]dioxol-4-ylmethyl (B)

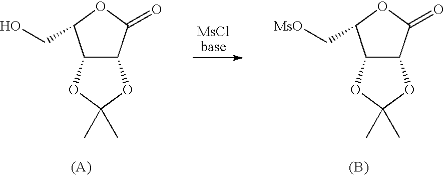

To a suspension of 2,3-O-isopropylidene-L-Lyxono-1,4-Lactone (A) (10 g, 53.1 mmol) and triethylamine (4.07 ml, 58.5 mmol) in CH₂Cl₂ (70 ml) was added a solution of methanesulfonyl chloride (4.52 ml, 58.5 mmol) in CH₂Cl₂ (20 ml). The addition was slow to maintain a temperature range between 0° C. and 10° C. The resulting mixture was stirred for 3 h at 25° C. and the resulting suspension was filtered. The cake was rinsed twice with 20 ml of CH₂Cl₂. The filtrate was washed twice with brine. The organic phase is concentrated on vacuum and 50 ml of hexane was added to precipitate the compound (B) and the mixture was cooled to −5 to 0° C. The solid was filtered and rinsed with 20 ml of hexane. The cake was dried under vacuum to give 14.1 g of (3aR, 4S, 6aR)-methanesulfonic acid 2,2-dimethyl-6-oxo-tetrahydro-furo[3,4-d][1,3]dioxol-4-ylmethyl (47.8 mmol, 90% yield). ¹H NMR 300 MHz (CDCl₃, δ ppm): 4.90 (s, 2H); 4.80 (m, 1H); 4.58 (dd, 1H); 4.48 (dd, 1H); 3.11 (s, 3H); 1.49 (s, 3H); 1.40 (s, 3H) ¹³C NMR: 172.49; 114.80; 76.21; 75.80; 75.36; 66.92; 37.76; 26.73; 25.80.

### Example 2

### Formation of (3aR, 4S, 6aR)-methanesulfonic acid 6-hydroxy -2,2,6-trimethyl-tetrahydro-furo[3,4-d][1,3]dioxol-4-ylmethyl ester (C)

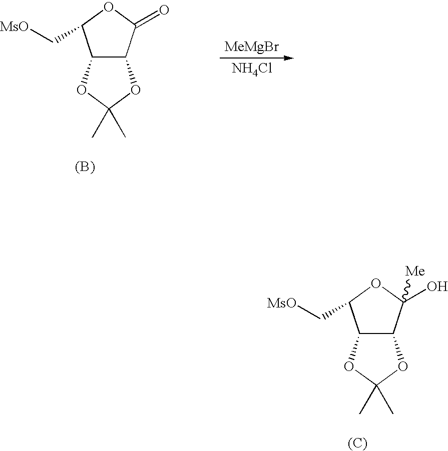

The (3aR, 4S, 6aR)-methanesulfonic acid 2,2-dimethyl-6-oxo-tetrahydro-furo [3,4-d][1,3]dioxol-4-ylmethyl (B) (10 g, 37.6 mmol) was suspended in THF (60 ml). The suspension was stirred at −10° C. to 0° C. and 29.6 mL of a solution of methylmagnesium bromide in THF/toluene (1.4M, 41.4 mmol) was added slowly to the solution while keeping the temperature below 0° C. The solution was stirred at 0° C. for 2 hours. The reaction was quenched with 50 ml of aqueous ammonium chloride, 10 ml of water and 60 ml of iPrOAc. The reaction mixture was stirred vigorously for one hour. The aqueous phase was separated and extracted with 30 ml of iPrOAc. The organic phases were combined and washed with 25 ml of brine and 25 ml of water. The organic phase was concentrated under vacuum and 60 ml of hexane was added to precipitate compound (C). The mixture was cooled to −10° C. to 0° C. The solid was filtered and washed with 20 ml of hexane. The cake was dried under reduced pressure to give 9.54 g of (3aR, 4S, 6aR)-methanesulfonic acid 6-hydroxy-2,2,6-trimethyl-tetrahydro-furo [3,4-d][1,3]dioxol-4-ylmethyl ester (C) (33.8 mmol, 90% yield). ¹H NMR 300 MHz (CDCl₃, δ ppm): 4.83 (m, 1H); 4.48 (m, 2H); 4.38 (m, 2H); 3.07 (s, 3H); 1.54 (s, 3H); 1.47 (s, 3H); 1.32 (s, 3H) ¹³C NMR: 112.92; 115.00; 85.18; 80.31; 76.41; 68.31; 37.44; 26.04; 24.69; 22.26.

### Example 3

### Formation of (3aR, 4S, 6aR)-2-(6-hydroxy-2,2,6-trimethyl-tetrahydro-furo [3,4-d][1,3]dioxol-4-ylmethyl)-isoindole-1,3-dione (D)

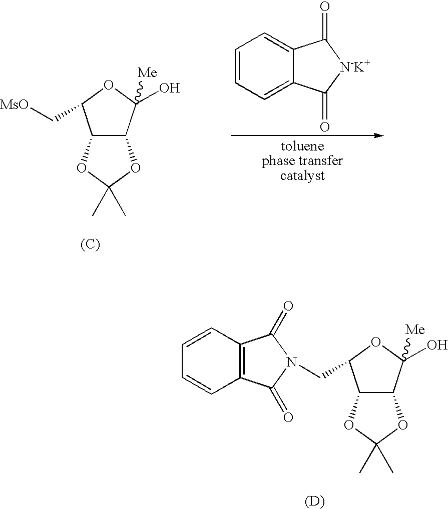

To a suspension of the (3aR, 4S, 6aR)-methanesulfonic acid 6-hydroxy -2,2,6-trimethyl-tetrahydro-furo[3,4-d][1,3]dioxol-4-ylmethyl ester (C) (10 g, 35.42 mmol) in 100 ml of toluene was added 8.53 g of potassium phthalimide (46.05 mmol) and 1.7 g of hexadecyltributylphosphonium bromide (3.5 mmol). The resulting mixture was refluxed with vigorous stirring for 6 h. The reaction temperature was cooled to room temperature and filtered on a Buchner funnel and washed with 20 ml of toluene. Water (2×10.0 ml) were added to the filtrate. The organic layer was separated and concentrated under vacuum. The crude was used directly in the next step. ¹H NMR 300 MHz (CDCl₃, δ ppm): 7.80 (dd, 2H); 7.68 (dd, 2H); 4.81 (dd, 1H); 4.45 (dt, 1H); 4.37 (d, 1H); 4.10 (dd, 1H); 3.82 (dd, 1H); 1.54 (s, 3H); 1.47 (s, 3H); 1.33 (s, 3H). ¹³C NMR: 167.94; 133.90; 133.69; 131.87; 123.20; 123.05; 112.85; 112.78; 105.01; 85.71; 80.72; 75.95; 37.45; 26.23; 25.16; 22.44.

### Example 4

### Formation of (3S, 7S, 7aR)-2,2,4-trimethyl-3a,6,7,7a-tetrahydro -[1,3]dioxolo[4,5-c]pyridin-7-ol (E)

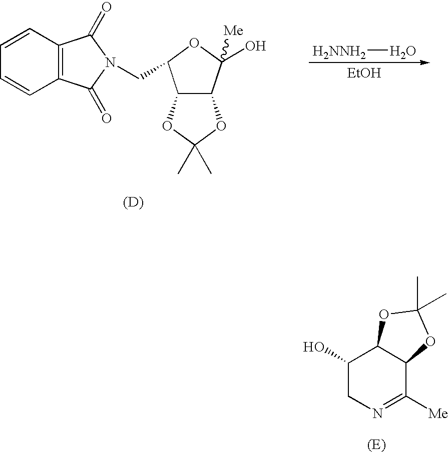

To a solution of (3aR, 4S, 6aR)-2-(6-hydroxy-2,2,6-trimethyl-tetrahydro-furo [3,4-d][1,3]dioxol-4-ylmethyl)-isoindole-1,3-dione (D) (10 g, 30.0 mmol) in EtOH (150 ml), was added 1.60 ml of hydrazine monohydrate (33.0 mmol). The resulting mixture was refluxed with vigorous stirring for 2 h. The reaction temperature was adjusted to 0° C. and the precipitate was vacuum filtered. The filtrate was passed on plug of silica gel to remove excess of hydrazine and the resulting (3S, 7S, 7aR)-2,2,4-trimethyl-3a,6,7,7a-tetrahydro-[1,3]dioxolo[4,5-c]pyridin-7-ol solution was used directly in the next step. ¹H NMR 300 MHz (CDCl₃, δ ppm): 4.38 (d, 1H); 4.22 (t, 1H); 3.78 (m, 2H); 3.40 (m, 1H); 2.13 (s, 3H); 1.41 (d, 6H). ¹³C NMR: 168.51; 109.69; 76.23; 72.91; 67.25; 51.82; 27.18; 25.42; 23.58.

### Example 5

### Formation of (3aS, 4R, 7S, 7aR)-2,2,4-trimethyl-1,3-dioxolo [4,5-c] piperidin-7-ol (F)

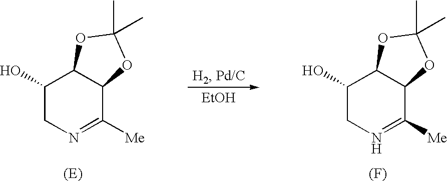

To the solution of the of (3S, 7S, 7aR)-2,2,4-trimethyl-3a,6,7,7a-tetrahydro-[1,3]dioxolo[4,5-c]pyridin-7-ol (E) (0.03 mole) in EtOH (150 ml) from step 4, was added 50% w/w of palladium on charcoal (50% w/w of water). The solution was charged to a hydrogenation reactor and was purged 3 times with hydrogen (45 psi) and then 45 psi of hydrogen was applied to the reactor. With vigorous stirring, the reaction temperature was then adjusted to 50° C. for 4 hrs. The reaction mixture was cooled to room temperature and the solution was filtered on filter aid. The filtrate was evaporated to give 4.78 g of a white to off-white crystalline compound (3aS, 4R, 7S, 7aR)-2,2,4-trimethyl-1,3-dioxolo [4,5-c] piperidin-7-ol (25.5 mmol, 850% step 4 and 5). ¹H NMR 300 MHz (CDCl₃, δ ppm): 4.02 (dd, 1H); 3.86 (dd, 1H); 3.64 (m, 1H); 3.10 (dd, 1H); 3.03 (dq, 1H); 2.42 (dd, 1H); 1.53 (s, 3H); 1.37 (s, 3H); 1.25 (d, 3H); 1.33. ¹³C NMR: 108.98; 80.77; 76.89; 71.38; 51.76; 48.94; 28.45; 26.50; 17.75.

Although the invention has been described in connection with certain preferred embodiments, it is not limited thereto. Rather, the invention includes all embodiments which may fall within the scope of the following claims.

1. A process for preparing a compound of the formula: 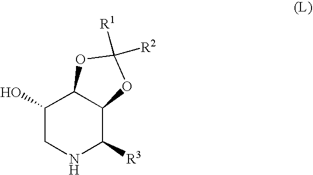
wherein R¹, R² and R³ are independently selected from the group comprising hydrogen, alkyl and alkoxy;
the process comprising:
(a) providing a compound of the formula: 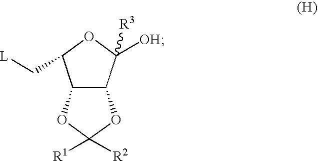
(b) reacting the compound of formula (H) with phthalimide to obtain a compound of the formula: 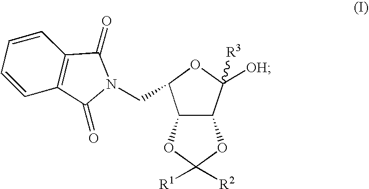
(c) cleaving the phthalimide group of compound (I) to obtain a compound of the formula: 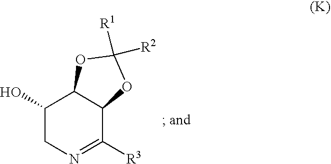
(d) hydrogenating the compound of formula (K) to obtain the compound of formula (L).

2. The process of claim 1, wherein the R¹, R² and R³ groups are selected from lower alkyl having 1 to 4 carbon atoms and lower alkoxy having 1 to 4 carbon atoms.

3. The process of claim 1, wherein R¹ and R² are methyl.

4. The process of claim 1, wherein R³ is methyl.

5. The process of claim 1, wherein R³ is methoxy.

6. A process for preparing a virus-inhibiting compound of the formula: 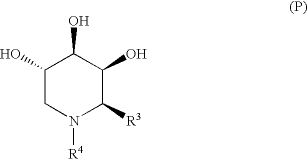
wherein R³ is selected from the group comprising hydrogen, alkyl and alkoxy, and R⁴ is a C₈ to C₁₆ alkyl or oxa-alkyl group;
the process comprising:
(a) providing a compound of the formula: 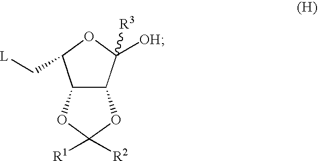
(b) reacting the compound of formula (H) with phthalimide to obtain a compound of the formula: 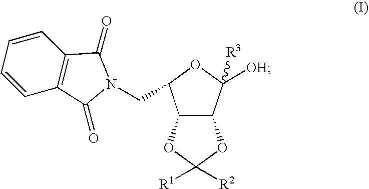
(c) cleaving the phthalimide group of compound (I) to obtain a compound of the formula: 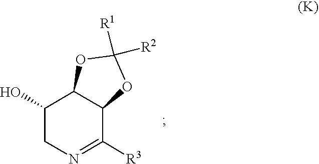
(d) hydrogenating the compound of formula (K) to obtain the compound of formula (L);
(e) N-alkylating and deprotecting the compound of formula (K) to obtain the compound of formula (P).
